(19) United States
(12) Patent Application Publication
(10) Pub. No.: US 2019/0110958 A1
(43) Pub. Date: Apr. 18, 2019

(54) SMART PACIFIER

(71) Applicant: William Joseph Addelia, II, Clinton Twp, MI (US)

(72) Inventor: William Joseph Addelia, II, Clinton Twp, MI (US)

(21) Appl. No.: 15/782,851

(22) Filed: Oct. 13, 2017

(51) Int. Cl. A61J 17/00; A61B 5/03; A61B 5/00

(52) U.S. Cl. CPC A61J 17/003; A61B 5/038; A61J 2200/72; A61B 5/746; A61B 5/486; A61B 5/0008

(57) ABSTRACT

A pacifier having a nipple portion and a handle portion separated by a collar. One or more sensors are disposed within the pacifier. The one or more sensors are configured to receive data from a user, wherein the user engages the nipple portion.

### CROSS-REFERENCE TO RELATED APPLICATION(S)

Not applicable

### BACKGROUND OF THE INVENTION

### 1. Field of Invention

The present invention relates to the field of child rearing devices, more particularly to oral soothing and enrichment devices for a child.

### 2. Description of Related Art

Pacifiers are often used to soothe or calm a child. Their shape is intended to simulate the shape of a nipple and stimulate the child into a rhythmic sucking motion against the pacifier. The suction caused from the sucking motion can act to create negative pressure against the child's gums. The rhythm can also create a subliminal cadence for the child to focus their attention away from crying to a fixation on the pacifier.

The design has remained rather constant where a pacifier is generally comprised of a nipple portion extending outward from a handle. The nipple portion is inserted into the child's mouth and a resilient material allows for the child to chew or bite on the nipple portion without resulting in damage.

Pacifiers are used with almost every single child for their ability to soothe the child. Accordingly, there has been little progress or innovation in supplemental function. The technology put into the design and structural components of current pacifiers is relatively rudimentary due to the singular purpose. However, the pervasive nature of their use would support the need for expanded elements allowing for a more dynamic utility.

One example of an attempt to expand on the function of a pacifier is U.S. Pat. No. 5,033,864 entitled “Temperature sensing pacifier with radio transmitter and receiver”. The temperature sensing elements disposed within the pacifier allows for indication of the child's temperature. While this example may provide for expansion beyond the capability to soothe the child, it fails to appreciate a number of other vital functions.

Based on the foregoing, there is a need in the art a device, having a dynamic and multi-use function in the configuration of a pacifier. That is, a device that will allow the least amount of disruption for the child and maximum amount of informational output to the parents.

### SUMMARY OF THE INVENTION

A pacifier having a nipple portion and a handle portion separated by a collar. One or more sensors are disposed within the pacifier. The one or more sensors are configured to receive data from a user, wherein the user engages the nipple portion.

In an embodiment, at least one of the one or more sensors is a temperature sensor, wherein the temperature sensor is configured to obtain a temperature of the user.

In an embodiment, at least one of the one or more sensors is a moisture sensor, wherein the moisture sensor is in communication with an exterior surface of the nipple portion, wherein the moisture sensor is configured to contact an interior of the user's mouth.

In an embodiment, the pacifier further comprises a wireless communication means electrically connected to a power source and at least one processor within the pacifier, wherein the one or more sensors are in communication with the processor, and wherein the processor is configured to compile data received from the one or more sensors.

In an embodiment, the pacifier wirelessly transmits data to a remote device, wherein the remote device controls operations of the one or more sensors within the pacifier.

In an embodiment, the pacifier further comprises a visual indicator disposed on the handle portion, wherein the visual indicator illuminates.

In an embodiment, the visual indicator is selectively initiated.

In an embodiment, the selective indication occurs when the pacifier becomes dislodges from an original position.

In an embodiment, one or more alerts are provided based on the data transmitted by the one or more sensors, wherein the one or more alerts are presented to the remote device through a display, wherein the alert is transmitted over one or more wireless networks.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a perspective view of the smart pacifier, according to an embodiment of the present invention;

FIG. 2 is a perspective view of the smart pacifier, according to an embodiment of the present invention;

FIG. 3 is a perspective view of the smart pacifier, according to an embodiment of the present invention;

FIG. 4 is a perspective view of the smart pacifier, according to an embodiment of the present invention;

FIG. 5 is a perspective view of the smart pacifier, according to an embodiment of the present invention;

FIG. 6 is a perspective view of the smart pacifier, according to an embodiment of the present invention;

FIG. 7 is a perspective view of the smart pacifier, according to an embodiment of the present invention.

### DETAILED DESCRIPTION OF PREFERRED EMBODIMENTS

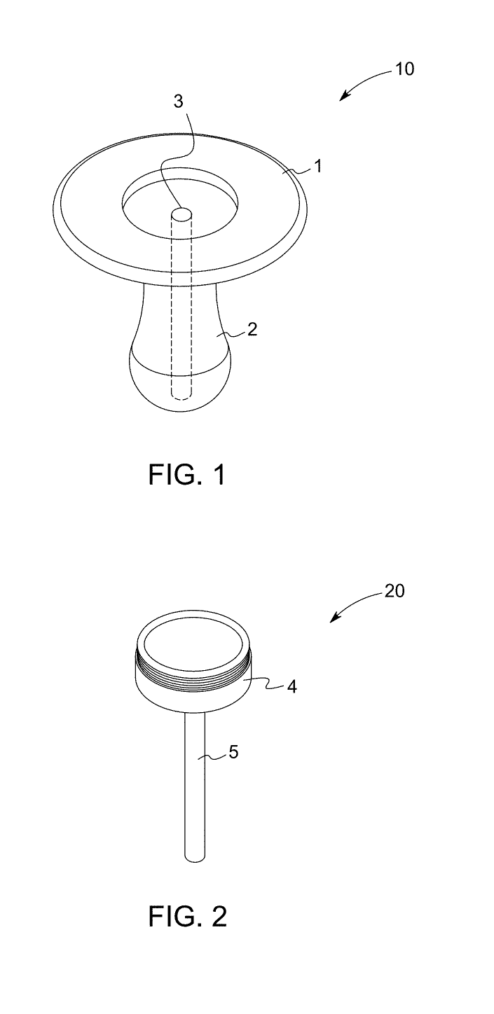

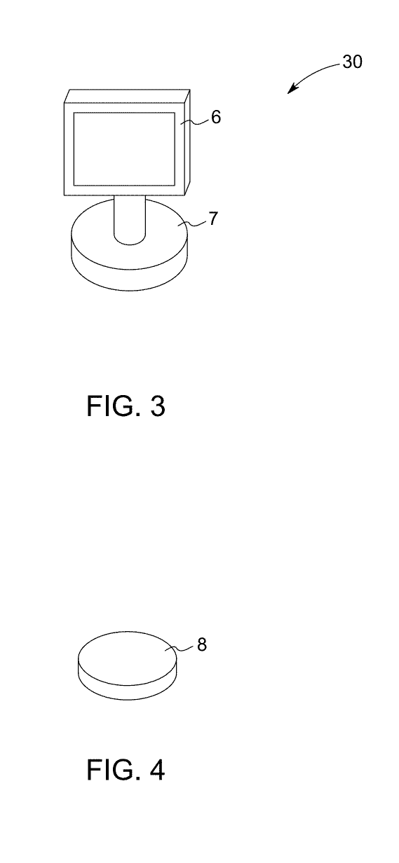

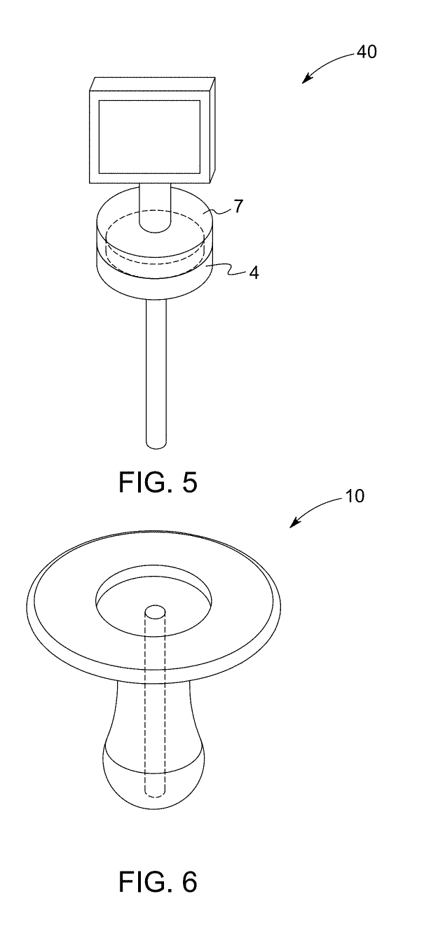

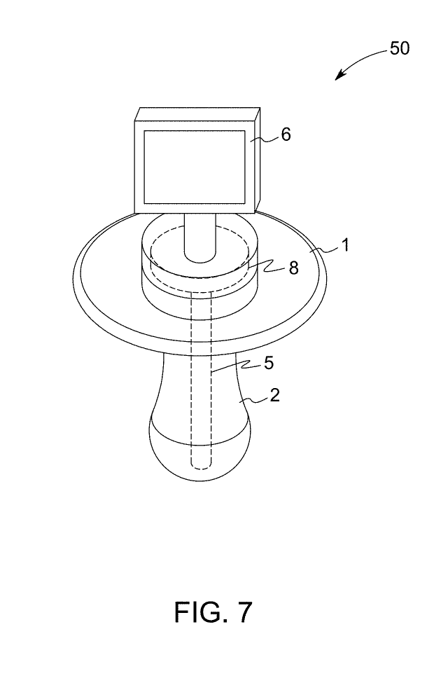

Preferred embodiments of the present invention and their advantages may be understood by referring to FIGS. 1-7.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

The present invention will now be described in detail with reference to embodiments thereof as illustrated in the accompanying drawings.

A smart pacifier is generally defined by a handle portion and a nipple portion. The nipple portion is in communication with the handle portion such that the handle portion is controlled by a parent when inserting the nipple portion into a mouth of a user. A collar may separate the nipple portion from the handle portion. The collar has a greater surface area than the nipple portion and the handle portion allowing the perimeter to extend a sufficient amount to prevent the handle portion from entering the user's mouth, when in use. A plurality of sensors are disposed within or on the pacifier. Each of the sensors is electrically connected to a power source and are each configured to provide information they receive.

One sensor may provide information relating to the temperature of the user. Such a temperature sensor may act as a thermometer and receive temperature data from contact or communication with the user's internal temperature.

In an embodiment, the temperature sensor is disposed within the nipple portion. A probe within the nipple portion receives temperature information from the inside of the user's mouth, when in use. The probe is then electrically connected to a visual indicator on an exterior surface of the handle portion. As the data is received by the probe sensor, the visual indicator provides visual and quantifiable temperatures to a separate person monitoring the visual indicator. In another embodiment, the probe sensor communicants with a remote device. For example, the remote device is a cellular phone and the probe sends the temperature data to the remote device. The visual indicator and information transmitted may provide for color indicators relating to threshold values of acceptable temperatures of the information received by the sensor. For example, a green, yellow, or red light may indicate rising levels of concern relating to the temperature.

In an embodiment, a glucose monitoring system is disposed within the device, whereby an effective amount of the user's saliva enters the glucose system through the nipple portion. The saliva contains a representative amount of blood glucose allowing the sample obtained to be processed within the device such that the blood glucose is determined and either displayed and/or transmitted to a remote user for consideration and monitoring.

In an embodiment, a respiratory monitoring system disposed within the device is configured to obtain data related to the breathing rate and deviations of the user. The nipple portion and/or the collar portion may be outfitted with a sensor configured to account for the number of breaths taken by the user. In another embodiment, the handle portion may have a sensor to receive respiratory information as it pertains to breathing through the user's nose. When exhaling or inhaling through the nose, the handle portion is equipped to sense rapid flow of air passing by it and relay the data to the central processing unit of the device for evaluation and monitoring.

In an embodiment, the device is configured to register and obtain the user's blood oxygen level. A light sensor appreciates the transmittance of light through one or more blood vessels. The oxygen sensor is in communication with the processor for analysis and monitoring.

In an embodiment, the device is configured to administer medication to the user. Medications may be preloaded into the device. The medications may be in the form of a solid, liquid, or gas depending on the prescription and medication being administered. The nipple portion will have one or more administration means disposed within or on the nipple portion. Where the medication would be a gas, one or more valves control dispensing of the medication through a vent system traversing the material of the nipple portion. A valve may control a metered flow depending on the volume of medication needed at a particular time. A specific example may be an inhaler that would introduce a gaseous steroid medication to the user depending on spikes in other biological data received. Where the medication is a solid or liquid, a dispensing mechanism allows for stored medication within the device to be dispersed outside of the device at preset intervals or on demand.

Preset intervals may be input into the processor and storage media of the device indicating and correlating with the desired dosage and administration times for the medication. The device will maintain records of the preset administration scheme and distribute the medication based on such inputs as triggered by an internal clock within the device providing a time for administration, or based on deviations in other data received from the device. A specific example would be the administration of insulin where the blood glucose levels of the user would require.

The temperature sensor may also work through infra-red (IR) temperature identification. An IR beam may be transmitted from a source disposed on the pacifier either at the nipple portion of on the collar. The beam contacts the skin of the user and receives temperature information as feedback.

In an embodiment, one of the sensors is a moisture sensor. Where health conditions manifest in a deviation of moisture of the interior of the user's mouth, the sensor detects shifts in the moisture level and relays them to a the visual indicator on the handle. In an embodiment, the visual indicator on the handle portion may provide for a liquid crystal display (LCD). The LCD is configured to provide real-time updated information in further detail than an objective scale of color or similar passive visual indicator system.

In an embodiment, one of the sensors is a blood pressure and heart rate monitor. A pressure sensitive sensor is disposed within or around the nipple portion substantially near the collar where the user's lips would contact the exterior of the nipple portion. Blood vessels in the lips dilate correspondingly with the user's pulse and a processor within the pacifier receives the quantity data obtained from the sensors and converts the data through known algorithms to a visual indicator, such as the LCD/LED display.

In an embodiment, the handle portions has a rotatable dial in communication with a handle protrusion extending in an opposite direction of the nipple away from the collar. The rotatable dial provides for a switch or selector to change between information generated by sensors within the pacifier. The rotatable dial may also serve as a timer where a user would rotate the dial to a predetermined position and the dial would rotate in a reverse order at a known speed based on a clock spring or ratchet mechanism configured to rotate at a known rate.

In an alternative embodiment, the rotatable dial is in communication with an audio producing means such as a speaker. In such an embodiment, the pacifier further comprises a storage media and a processor in communication with the speaker and the dial. The dial may be rotated to select an audio file within the storage media. The audio file would be executed by the processor and transmitted from the speaker as a sound. For example, a person may rotate the dial to a lullaby, which will play for a predetermined amount of time.

The handle portion may also have a projection means. A light source may project light outward from the handle portion. One or more buttons may be in communication with the processor and the storage media. Once the button is engaged, the processor will execute instructions for displaying of different colors, patterns, or other attributes of a projected light from a light source in the handle. For example, a specific color may be selected and projected from the light source throughout the room creating ambient light for the user. Patterns may include shapes or know figures such as animals or stars, which are projected onto a surface away from the handle portion. In some example, the patterns may more based on the instructions executed by the processor.

In an embodiment, one of the sensors is an engagement sensor. The sensor acts as a trigger to provide for visual indication of the pacifier in the event it should become dislodges from the user. The sensor may be activated upon an initial contact with the user. In such an example, one or more sensors may be disposed on the collar surface contacting the users skin, or on/within the nipple portion so long as a positive contact with the user is triggered. The sensor may use temperature or moisture content of the surrounding environment of the pacifier for triggering of the switch. Once triggered, the switch remains in an active state so long as the heat or moisture data does not deviate from the triggering baseline value. When the data does deviate from such a value, a visual indicator is provided to facilitate rapid location of the pacifier. For example, the visual indicator may be a portion of the handle that illuminates when the trigger is no longer in the activated position. In an embodiment, when the trigger is disengaged from the active state, an alert may be sent to a remote device to indicate the user is no longer in contact with the pacifier.

In an embodiment, a system operating within the mobile device is configured to receive, interpret, and display the received data. A graphical user interface allows for interaction between the system and the user for selective display or data requests. The user engages the system through the graphical user interface and may select one or more options relating to available data being transmitted by the sensors within the pacifier.

In another embodiment, where the sensors transmit data to the remote device, threshold values may be established within the remote device relating to medically appreciated base-line expectations for the values of the data transmitted. Where the remote device receives data outside of the threshold values, an alert system may be triggered providing a notification to the remote device and supplying information relating to the reason for the alert. The alert may be escalated as necessary to emergency service personnel for their consideration. In yet another embodiment, special profiles are created within the system and distributed to selected or necessary remote devices. The profiles may reflect unique medical conditions of the user allowing for more proactive monitoring by the remote device, of the data being transmitted from the sensors.

The remote device may control operations of the pacifier remotely. For example, data may be uploaded to the storage media within the pacifier and recalled upon transmission of an initiation signal sent to the pacifier from the remote device. For example, parents may pre-record words, phrases, or sounds to the pacifier and may remotely initiate playback to the user at any time. A specific example would be the sound of a parents voice to be remotely triggered in the event the heart rate elevates or the pacifier contact trigger is deactivated. Such an example would allow for a soothing of the child by the playback of the audio while the parent was allowed time to go a replace the pacifier.

In an embodiment, the pacifier is wirelessly connected to one or more remote computing devices. The pacifier may comprise a Bluetooth® or similar transmission means for the wireless transmission and receipt of data between the pacifier and the remote device.

In some embodiments, the pacifier may have an attachment means configured to create a positive engagement between the pacifier and the user or a separate surface. The attachment prevents the loss or negative contact between the pacifier and a contaminated surface. The attachment means may be a clip or fastener separated from the pacifier handle portion by a strap or tether.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A pacifier comprising:
a. a nipple portion and a handle portion separated by a collar;
b. one or more sensors disposed within the pacifier, wherein the one or more sensors are configured to receive data from a user, wherein the user engages the nipple portion.

2. The pacifier of claim 1, wherein at least one of the one or more sensors is a temperature sensor, wherein the temperature sensor is configured to obtain a temperature of the user.

3. The pacifier of claim 1, wherein at least one of the one or more sensors is a moisture sensor, wherein the moisture sensor is in communication with an exterior surface of the nipple portion, wherein the moisture sensor is configured to contact an interior of the user's mouth.

4. The pacifier of claim 1, further comprising a wireless communication means electrically connected to a power source and at least one processor within the pacifier, wherein the one or more sensors are in communication with the processor, and wherein the processor is configured to compile data received from the one or more sensors.

5. The pacifier of claim 1, wherein the pacifier wirelessly transmits data to a remote device, wherein the remote device controls operations of the one or more sensors within the pacifier.

6. The pacifier of claim 1, further comprising a visual indicator disposed on the handle portion, wherein the visual indicator illuminates.

7. The pacifier of claim 6, wherein the visual indicator is selectively initiated.

8. The pacifier of claim 7, wherein the selective indication occurs when the pacifier becomes dislodges from an original position.

9. The pacifier of claim 5, wherein one or more alerts are provided based on the data transmitted by the one or more sensors, wherein the one or more alerts are presented to the remote device through a display, wherein the alert is transmitted over one or more wireless networks.
